(19) United States
(12) Patent Application Publication
(10) Pub. No.: US 2016/0287835 A1
(43) Pub. Date: Oct. 6, 2016

(54) METHOD FOR TREATING ANGINA

(71) Applicant: Nogina, LLC, Columbia, SC (US)

(72) Inventor: Ezra B. Riber, Columbia, SC (US)

(21) Appl. No.: 15/090,131

(22) Filed: Apr. 4, 2016

Related U.S. Application Data
(60) Provisional application No. 62/142,055, filed on Apr. 2, 2015.

(51) Int. Cl. A61M 19/00; A61M 25/01; A61M 31/00

(52) U.S. Cl. CPC A61M 19/00; A61M 31/00; A61M 25/0102; A61M 2210/0618

(57) ABSTRACT

A method for treating angina by introducing medicine to a patient's sphenopalatine ganglion with a catheter. The medicine includes a mixture of a local anesthetic, a steroid, an analgesic and an anti-inflammatory.

### CROSS-REFERENCE TO RELATED APPLICATION

This application claims the benefit of U.S. Provisional Patent Application No. 62/142,055, filed Apr. 2, 2015, which is entirely incorporated herein by reference.

### TECHNICAL FIELD

This invention relates to cervico-thoracic sympathetic blockade via blockade of the sphenopalatine ganglion.

### BACKGROUND

Chest pain often occurs because of vasoconstriction of the vessels to the heart. This can be chronic narrowing as in atherosclerosis or physiological as occurs with vasospasm. Syndrome X is one example of a refractory anginal condition where patients have vasoconstriction of blood vessels causing severe debilitating chest pain. In these patients, the angina is due to endothelial microvascular dysfunction. Physicians sometimes treat Syndrome X patients with multiple and oftentimes high doses of anti-anginal medications, both maintenance and abortive. It is not uncommon for these anti-anginal medications to provide sporadic relief at best.

A stellate ganglion block or cervic-thoracic sympathetic block, carried out via injection administered in the low cervical spine or upper thoracic spine, can cause vasodilation, thereby increasing blood flow to the heart muscle itself. A stellate ganglion block can be utilized to break the chest pain cycle associated with Syndrome X and other refractory angina conditions. In some institutions, the stellate ganglion block procedure, if beneficial, may be followed by placement of a spinal cord stimulator in the thoracic spine. The placement of this stimulator is a major undertaking because it includes spine surgery with costs exceeding one hundred thousand dollars. Treatment options for patients experiencing angina include multiple medications with variable effectiveness and potential side effects, limiting physical activity, and repeated and costly emergency room visits, and hospitalization.

As a result, an improved treatment for angina that is less intrusive and more effective is desirable.

### SUMMARY

Utilizing a sphenopalatine ganglion block method to treat Syndrome X and refractory angina in general as a substitute for an invasive stellate ganglion block has yielded surprising and unexpected results. Patients with Syndrome X, as well as those with angina secondary to coronary artery disease, that have been treated with the sphenopalatine ganglion block method have remarkable extended pain relief. Many times, the pain relief lasts for months. Another benefit of the sphenopalatine ganglion block method is the reduction of maintenance and abortive medication while the patient's activity level increases following the administration of a sphenopalatine ganglion block.

In a first aspect, the present disclosure relates to a method for treating angina. The method includes inserting a catheter having a straightening member into the nostril of a patient. The catheter has an insertion end and a manipulation end. The insertion end has an intrinsic curvature with respect to the longitudinal axis of the catheter and the straightening member is disposed within the catheter. The intrinsic curvature conforms to a patient's nasal anatomy such that the catheter is insertable into the sphenopalatine recess of the patient. The method also includes advancing the catheter with the straightening member inserted into the catheter past the middle sinus turbinate within the nasal cavity of the patient. The method also includes removing the straightening member from the catheter so that the catheter bends in a direction toward the patient's sphenopalatine recess. The method also includes advancing the catheter into the patient's sphenopalatine recess. The method also includes dispensing a mixture of medication to the patient's sphenopalatine ganglion disposed within the sphenopalatine recess of the patient.

In another aspect, the present disclosure relates to a method for treating angina by introducing medicine to a patient's sphenopalatine ganglion with a catheter. The medicine includes a mixture of a local anesthetic, a steroid, an analgesic and an anti-inflammatory.

In still another aspect, the present disclosure relates to a solution for delivery to a sphenopalatine ganglion to treat angina. The solution includes between about 64% to about 83% of a local anesthetic, between about 8% to about 13% of a steroid, between about 4% to about 10% of an analgesic, and between about 0.07% to about 2.0% of an anti-inflammatory.

### DESCRIPTION OF EXAMPLE EMBODIMENTS

Language referring to the features and advantages is understood to mean that a specific feature, advantage, or characteristic described in connection with an embodiment is included in at least one embodiment of the present invention. Thus, discussion of the features and advantages, and similar language, throughout this disclosure may, but do not necessarily, refer to the same embodiment.

The sphenopalatine ganglion is a collection of neurons that reside within the pterygopalatine fossa and is innervated by the maxillary division of the trigeminal nerve. A sphenopalatine ganglion blockade is a minimally invasive procedure used to treat chronic migraine headaches and facial pain.

A sphenopalatine ganglion block can be administered via a catheter-like medical device (“SPG Block Device”) for facilitating intranasal administration of a medication to a patient's pterygopalatine fossa (“SPG Method”). Such SPG Block Devices are described in U.S. Pat. No. 8,388,600, hereinafter incorporated by reference, sold by Dolor Technologies of Salt Lake City, Utah under the trade name Sphenocath™, or alternatively by Medical Components, Inc. under the trade name Allevio™ SPG Nerve Block Catheter.

An example SPG Block Device can include a catheter having a straightening member into the nostril of a patient. The catheter further includes an insertion end and a manipulation end. The insertion end can have an intrinsic curvature with respect to the longitudinal axis of the catheter and the straightening member is disposed within the catheter. The intrinsic curvature conforms to a patient's nasal anatomy such that the catheter is insertable into the sphenopalatine recess of the patient

Through trials administering certain medications using the SPG device, the relief is immediate as opposed to the indeterminate and variable onset of medication effectiveness, if any relief is obtained at all. Additionally, there are no appreciable side effects as compared with those associated with cardiac medications, all of which have some significant potential side effects.

A method of the present invention is presented for treating refractory angina unresponsive to conventional pharmacological intervention. The method in the disclosed embodiments substantially includes the steps necessary to carry out the functions presented above with respect to the operation of the described apparatus.

To perform the SPG Method, a patient is placed in a supine position. The supine position is optimal for saturating the ganglion with the injectate through gravitational force as the properly positioned catheter just superior to the middle turbinate directs the injectate to flow posteriorly (“downward”) saturating the pterygopalatine fossa.

The SPG Block Device is inserted into the nasal cavity of a patient lying in a supine position at a depth sufficient to pass the middle turbinate. Once the SPG Block Device is advanced toward the Pterygopalatine Fossa, the physician uses fluoroscopic imaging to ensure the SPG Block Device is placed in the appropriate position so as to allow the flow of medication toward the Pterygopalatine Fossa. Contrast solution (X-ray dye), visible on fluoroscopic imaging, is used to confirm the flow of the medication. The volume range of injected contrast is 0.5 to 3.0 ml, but typically 1.0 ml. However one skilled in the art would appreciate the amounts necessary to confirm the SPG Block Device's placement in close proximity to the target site.

A syringe containing the medication is connected to the SPG Block Device. Once the target site is confirmed, the syringe injects a medication into and through the SPG Block Device which will flow towards pterygopalatine fossa. The patient will remain supine for up to 10 minutes after application of the medication.

In all embodiments, the method administers a mixture of medication to the sphenopalatine ganglion located within the pterygopalatine fossa of the patient. The mixture can introduce various medications in each nostril. The total amount of the mixture by volume is preferably between about 2.0 ml and about 2.5 ml bilaterally (i.e. per nostril), preferably between about 2.1 ml and about 2.4 ml, most preferably about 2.25 ml. The mixture can be drawn up into a 5 cc syringe, which delivers the mixture to the nostrils through the SPG Block Device towards the pterygopalatine fossa.

The mixture per nostril can include an amount of a local anesthetic, for example Lidocane, more specifically a 4% (concentration) Lidocane, in an amount ranging between at least 2.0-2.5 mL, preferably about 2.0 ml. This amount of local anesthetic represents at least between about 64% to about 83%, most preferably about 80%, of the total amount of the mixture. These defined volumes and percentages were established based on the inventor's expertise and produced unexpectedly successful results.

The mixture per nostril can also include an amount of a steroid cortisone, for example Betamethasone in a concentration of 6 mg/ml, in an amount ranging between at least 1.5 mg (i.e., 0.25 ml) to 2.0 mg (0.33 ml), preferably about 2.0 mg. This amount of local anesthetic represents between at least about 8% to about 13%, most preferably about 10%, of the total amount of the mixture. These defined volumes and percentages were established based on the inventor's expertise and produced unexpectedly successful results.

The mixture per nostril can also include an amount of an herbal analgesic derived from the Pitcher plant, for example Sarapin, in an amount ranging between at least 0.125 ml to 0.25 ml, preferably about 0.25 ml. This amount of herbal analgesic represents between at least about 4% to about 10%, most preferably about 8%, of the total amount of the mixture. These defined volumes and percentages were established based on the inventor's expertise and produced unexpectedly successful results.

The mixture per nostril can also include an amount of a nonsteroidal anti-inflammatory, for example Ketorolac in a concentration of 60 mg/ml, in an amount ranging between at least 1.5 mg (i.e., 0.025 ml) to about 3.0 mg (i.e., 0.05 ml), preferably about 1.5 mg. This amount of nonsteroidal anti-inflammatory represents between at least about 0.07% to about 2.0%, most preferably at least about 1% -2%, of the total amount of the mixture. These defined volumes and percentages were established based on the inventor's expertise and produced unexpectedly successful results.

Other medications may be used in combinations to result in desired effects. Flavorings may also be utilized to make the medication mixture more palatable.

The SPG Block device can be utilized without the benefit of fluoroscopy, such that a blind technique or non-fluoroscopic technique to deliver the medication is used as quickly as possible in an effort to stabilize the person experiencing the anginal symptoms.

By influencing the sympathetic chain of the superior cervical ganglion leading to T1 and T2, sphenopalatine ganglion blockade with the proprietary liquid solution can decrease chronotropy and inotropy, thereby leading to improvement in angina symptoms in adult patients who have persistent symptomatic non-obstructive atherosclerosis.

By blocking the sympathetic innervation of the vascular adventitia, sphenopalatine ganglion blockade with the proprietary liquid solution decreases the amount of vasoconstriction of the arteries and veins thereby leading to decreased coronary microvascular resistance and vasospasm in adult patients who have persistent symptomatic non-obstructive atherosclerosis.

Six (6) patients with syndrome X and intractable angina have had successful relief of chest pain and increase in activity levels for months at a time following treatment through the method described above. See chart below.

| | Percent | Duration | | |
|---|---|---|---|---|
| No | Relief | of Relief | Reproducibility | Comments |
| 1 | 85-100% | 2-3 months | x4 | Improved functioning |
| 2 | 75-90% | 3-4 months | x6 | Improved function |
| | | | | and O2 sat |
| 3 | 90-100% | 3-5 months | x8 | Improvement even with |
| | | | | knee rehab |
| 4 | 75-80% | 1-2 months | x4 | Also has CAD |
| 5 | 90-100% | Ongoing | x1 | Improved functioning |

Although specific embodiments of the disclosure have been described, numerous other modifications and alternative embodiments are within the scope of the disclosure. For example, any of the functionality described with respect to a particular device or component may be performed by another device or component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

We claim:

1. A method for treating angina the, method comprising:
inserting a catheter having a straightening member into the nostril of a patient, the catheter further comprising an insertion end and a manipulation end, the insertion end having an intrinsic curvature with respect to the longitudinal axis of the catheter and the straightening member is disposed within the catheter, the intrinsic curvature conforming to a patient's nasal anatomy such that the catheter is insertable into the sphenopalatine recess of the patient;
advancing the catheter with the straightening member inserted into the catheter past the middle sinus turbinate within the nasal cavity of the patient;
removing the straightening member from the catheter whereby the catheter bends in a direction toward the patient's sphenopalatine recess;
advancing the catheter into the patient's sphenopalatine recess; and
dispensing a mixture of medication to the patient's sphenopalatine ganglion disposed within the sphenopalatine recess of the patient.

2. The method of claim 1, further comprising identifying a direction of the intrinsic curvature and aligning the intrinsic curvature of the catheter with the patient's sphenopalatine recess.

3. The method of claim 1, further comprising identifying a defined depth of the catheter, the defined depth comprising a depth equaling a distance between an entrance to a patient's sphenopalatine recess and an external entrance to the patient's nostril.

4. The method of claim 3, wherein medication is delivered over the middle sinus turbinate to achieve sphenopalatine ganglion blockade.

5. The method of claim 4, wherein the sympathetic nerves of the patient are impacted by accessing the sphenopalatine recess at the back of the sinus cavity extending from the top of the middle sinus turbinate.

6. The method of claim 3, wherein transnasal sphenopalatine ganglion blockade is achieved by delivery of medicine over the middle sinus turbinate.

7. The method of claim 1, wherein the mixture of medication comprises a local anesthetic, a steroid, an analgesic and an anti-inflammatory.

8. The method of claim 7, wherein the local anesthetic comprises Lidocane.

9. The method of claim 7, wherein the steroid comprises cortisone.

10. The method of claim 7, wherein the analgesic comprises is herbal.

11. The method of claim 7, wherein the anti-inflammatory is nonsteroidal.

12. The method of claim 7, wherein the mixture of medication comprises between about 64% to about 83% of local anesthetic, between about 8% to about 13% of steroid, between about 4% to about 10% of analgesic, and between about 0.07% to about 2.0% of anti-inflammatory.

13. The method of claim 7, wherein the mixture of medication comprises between about 2.0 ml to about 2.5 ml of local anesthetic, between about 0.25 ml to about 0.33 ml of steroid, between about 0.125 ml and about 0.25 ml of analgesic, and between about 0.025 ml and about 0.05 ml of anti-inflammatory.

14. A method for treating angina comprising introducing medicine to a patient's sphenopalatine ganglion with a catheter, the medicine comprising a mixture of a local anesthetic, a steroid, an analgesic and an anti-inflammatory.

15. The method of claim 14, further comprising confirming a medicine-introduction site with fluoroscopic imaging.

16. The method of claim 14, further comprising introducing fluoroscopic imaging contrast solution to confirm the flow of the medication

17. The method of claim 14, wherein the mixture of medication comprises between about 64% to about 83% of local anesthetic, between about 8% to about 13% of steroid, between about 4% to about 10% of analgesic, and between about 0.07% to about 2.0% of anti-inflammatory.

18. The method of claim 14, wherein the mixture of medication comprises between about 2.0 ml to about 2.5 ml of local anesthetic, between about 0.25 ml to about 0.33 ml of steroid, between about 0.125 ml and about 0.25 ml of analgesic, and between about 0.025 ml and about 0.05 ml of anti-inflammatory.

19. A solution for delivery to a sphenopalatine ganglion to treat angina, the solution comprising between about 64% to about 83% of a local anesthetic, between about 8% to about 13% of a steroid, between about 4% to about 10% of an analgesic, and between about 0.07% to about 2.0% of an anti-inflammatory.
